(19) United States
(12) Patent Application Publication
(10) Pub. No.: US 2022/0096199 A1
(43) Pub. Date: Mar. 31, 2022

(54) Medical Marker

(71) Applicant: NIPPON PISTON RING CO., LTD., Saitama City (JP)

(72) Inventors: Takasumi KUBO, Saitama (JP); Masahiro SHINZAWA, Saitama (JP); Yuki KIMURA, Saitama (JP); Mayuki ARAI, Saitama (JP)

(21) Appl. No.: 17/487,154

(22) Filed: Sep. 28, 2021

(30) Foreign Application Priority Data
Sep. 29, 2020 (JP) 2020-163778

(51) Int. Cl. A61B 90/00

(52) U.S. Cl. CPC A61B 90/39

(57) ABSTRACT

A medical marker is provided that will remain at a predetermined site in a patient's body. The medical marker includes a helical body obtained by making a linear body in a helical shape. When the helical body is viewed from an axial direction of the helical body, a winding shape of a unit winding constituting one winding of the helical body is a non-perfect circle, and a plurality of the unit windings contiguously aligned in the axial direction overlap at a terminal end part of one of the adjacent unit windings and a start end part of the other of the adjacent unit windings. As a result, there is a phase difference in the winding shape by an amount of overlap.

### CROSS REFERENCE TO RELATED APPLICATIONS

The present application is based on, and claims priority from JP Application Serial Number 2020-163778, filed Sep. 29, 2020, the disclosure of which is hereby incorporated by reference herein in its entirety.

### BACKGROUND

### Technical Field

The present invention relates to a medical marker that will remain as a mark at a predetermined site in a living body.

### Related Art

Conventionally, as an intrusive marker that will remain as a mark at a predetermined site in a living body, a marker has been proposed that has the form of a helical coil having segments with different pitches, is flexible in axial and lateral directions, and is deformable so as to follow changes in the shape, size, or position of an anatomical site (refer to, for example, Japanese Patent No. 5629698). Since the intrusive marker is deformable so as to follow changes in the shape, size, or position of the anatomical site, the marker can stay at a predetermined site in a patient's body.

The above-described intrusive marker is a helical coil having a moderately uneven surface. The above-described intrusive marker remains at a predetermined site in a patient's body with the uneven surface in close contact with the site. Therefore, the anchoring effect of the above-mentioned intrusive markers to remain at the predetermined site in the patient's body is weak. As a result, it cannot be expected that the above-described intrusive marker stably remains at the predetermined site in the patient's body.

Considering such circumstances, the present invention aims at providing a medical marker that can stably remain at a predetermined site in a patient's body.

### SUMMARY

A medical marker according to the present invention is a medical marker including a helical body obtained by making a linear body in a helical shape, wherein when the helical body is viewed from an axial direction of the helical body, a winding shape of a unit winding constituting one winding of the helical body is a non-perfect circle, and a plurality of the unit windings contiguously aligned in the axial direction overlap at a terminal end part of one of the adjacent unit windings and a start end part of the other of the adjacent unit windings. As a result, there is a phase difference in the winding shape by an amount of overlap.

In the medical marker according to the present invention, the winding shape of the unit winding has a vertex region with a locally reduced curvature.

In the medical marker according to the present invention, the winding shape of the unit winding is a polygon with a plurality of the vertex regions or a crushed perfect circle.

In the medical marker according to the present invention, the vertex regions of the plurality of unit windings contiguously aligned in the axial direction are each arranged so as to sequentially shift to one side of a circumferential direction of the helical body along one side of the axial direction.

In the medical marker according to the present invention, when the helical body is viewed from the axial direction, an end winding shape of an end unit winding constituting one winding of the helical body at least at one end in the axial direction is different from the winding shape of the unit winding in the middle of the helical body in the axial direction.

In the medical marker according to the present invention, the end winding shape is a perfect circle.

In the medical marker according to the present invention, when the unit winding is in the shape of a regular polygon, the phase difference θ and a central angle α of the regular polygon satisfy 0.1α≤θ≤0.9α.

In the medical marker according to the present invention, when the unit winding is in the shape of a regular polygon, a maximum strain εₛ of a material constituting the helical body, a radius R of a circumscribing circle of the regular polygon, a radius ω of the linear body, and the number n of corners of the regular polygon satisfy the following mathematical formula.

$${ɛ}_{s} ≧ 100 \times \{ \frac{R \times sin ( \frac{360 °}{2 n} ) \times n}{R \times sin ( \frac{360 °}{2 n} ) \times n - πω} - 1 \} \div n$$

A method for manufacturing a medical marker according to the present invention is a method for manufacturing the medical marker described above, the method including forming the helical body by springing back, after the linear body is wound around a columnar core material.

A method for manufacturing a medical marker according to the present invention is a method for manufacturing the medical marker described above, the method including forming the helical body by springing back, after the linear body is formed into a coil using a coiling device for forming the linear body into the coil.

In the method for manufacturing the medical marker according to the present invention, the helical body is shaped by heat treatment after being spring-backed.

A medical marker according to the present invention is a medical marker including a helical body obtained by making a linear body in a helical shape, wherein when the helical body is viewed from an axial direction of the helical body, at least one of a plurality of unit windings, each of which constitutes one winding of the helical body and which are contiguously aligned in the axial direction, has a convex portion that is convex outwardly in a direction orthogonal to the axial direction with reference to an outer edge of adjacent one of the unit windings.

### Advantageous Effects of Invention

According to the medical marker of the present invention, it is possible to achieve the superior effect of stably remaining at a predetermined site in a patient's body.

### BRIEF DESCRIPTION OF THE DRAWINGS

FIG. 1 is a perspective view of a medical marker according to a first embodiment of the present invention;

FIG. 2A is a plan view of the medical marker according to the first embodiment of the present invention;

FIG. 2B is a side view of the medical marker according to the first embodiment of the present invention;

FIG. 3A is a perspective view of a linear body and a core material used in manufacturing the medical marker according to the first embodiment of the present invention, FIG. 3B is a side view illustrating a state in which the linear body is wound around the core material, and FIG. 3C is a plan view of a precursor, which is formed by winding the linear body around the core material, according to the first embodiment of the present invention;

FIG. 4A is a simplified plan view illustrating the precursor, which is formed by winding the linear body around the core material, according to the first embodiment of the present invention, and FIGS. 4B and 4C are simplified plan views illustrating a part of the medical marker according to the first embodiment of the present invention;

FIGS. 5A and 5B are simplified plan views illustrating a part of the medical marker according to the first embodiment of the present invention;

FIG. 6A is a perspective view illustrating a linear body and a core material used in manufacturing a modification of the medical marker according to the first embodiment of the present invention, FIG. 6B is a side view illustrating a state in which the linear body is wound around the core material, and FIG. 6C is a plan view of a modification of the precursor, which is formed by winding the linear body around the core material, according to the first embodiment;

FIG. 7A is a plan view of another modification of the medical marker according to the first embodiment of the present invention, and FIG. 7B is a side view of the modification of the medical marker according to the first embodiment of the present invention;

FIGS. 8A and 8B are plan views of other modifications of the medical marker according to the first embodiment of the present invention;

FIG. 9A is a plan view illustrating a star-shaped core material used in manufacturing yet another modification of the medical marker according to the first embodiment of the present invention, and FIG. 9B is a plan view of yet another modification of the precursor according to the first embodiment of the present invention;

FIG. 10A is a plan view of a medical marker according to a second embodiment of the present invention, and FIG. 10B is a side view of the medical marker according to the second embodiment of the present invention; and

FIG. 11 is a side view of a modification of the medical marker according to the second embodiment of the present invention.

### DETAILED DESCRIPTION

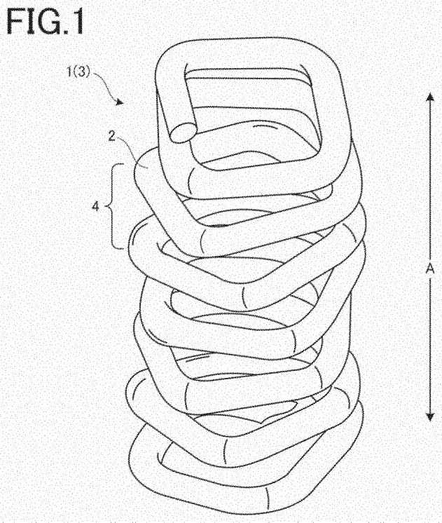

Embodiments of the present invention will be described below with reference to the accompanying drawings. FIGS. 1 to 11 illustrate examples of the embodiments of the invention, and in the drawings, components denoted by the same reference numerals as in FIG. 1 represent the same components.

### First Embodiment

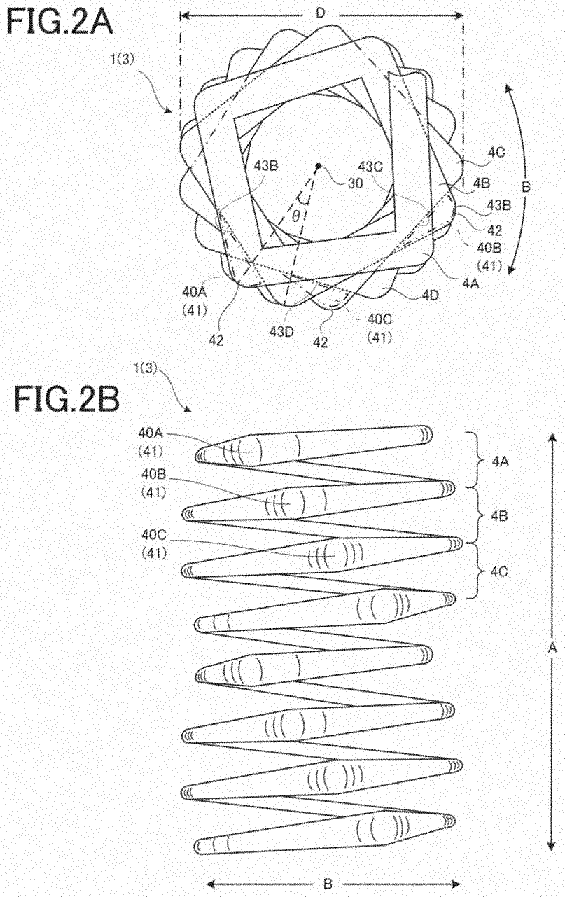

A medical marker 1 according to a first embodiment of the present invention is inserted into a living body, for example, by placing the medical marker 1 in an insertion needle and pushing the medical marker 1 out, to place the medical marker 1 at a predetermined site in the living body. The medical marker 1 remains as a mark at the predetermined site in the living body. As illustrated in FIGS. 1 to 2B, the medical marker 1 according to the present embodiment includes a helical body 3 obtained by making a linear body 2 in a helical shape. The helical body 3 includes a plurality of unit windings 4 each of which constitutes one winding of the helical body 3. The plurality of unit windings 4 are contiguously aligned in an axial direction A of the helical body 3 (hereinafter the axial direction of the helical body 3 will be referred to simply as the axial direction.).

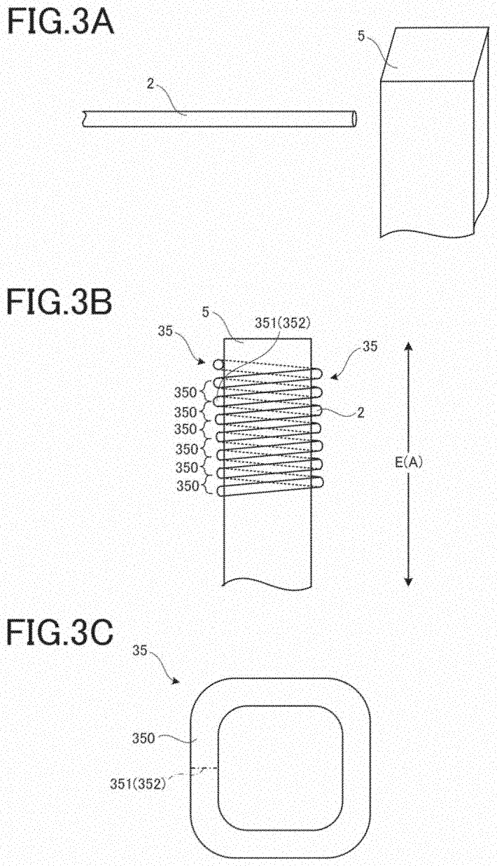

To manufacture the medical marker 1, as illustrated in FIG. 3A, a core material 5, which is a columnar material with a square cross-sectional shape, and the linear body 2 are prepared. As illustrated in FIG. 3B, the linear body 2 is wound around the core material 5 in a helical shape. In this state, one winding of the linear body 2 (precursor unit winding 350) is formed into the square columnar shape to make a precursor 35 of the helical body 3. The precursor 35 of the helical body 3 may be made by forming the linear body 2 into a coil shape using a coiling device (not illustrated), instead of winding the linear body 2 around the core material 5. The coiling device can form the linear body 2 into a coil. Examples of the coiling device may include a device adopting a method with a coiling pin without a core material 5. However, the coiling device is not limited to using this method and may use a different method. As illustrated in FIG. 3C, the precursor 35 has a square shape, when the helical body 3 is viewed from the axial direction A. By removing the core material 5 from the precursor 35, the precursor 35 springs back in its own circumferential direction B and becomes the helical body 3 as illustrated in FIGS. 1 to 2B. In order to stabilize the shape of the helical body 3, heat treatment may be applied to the helical body 3 for shaping.

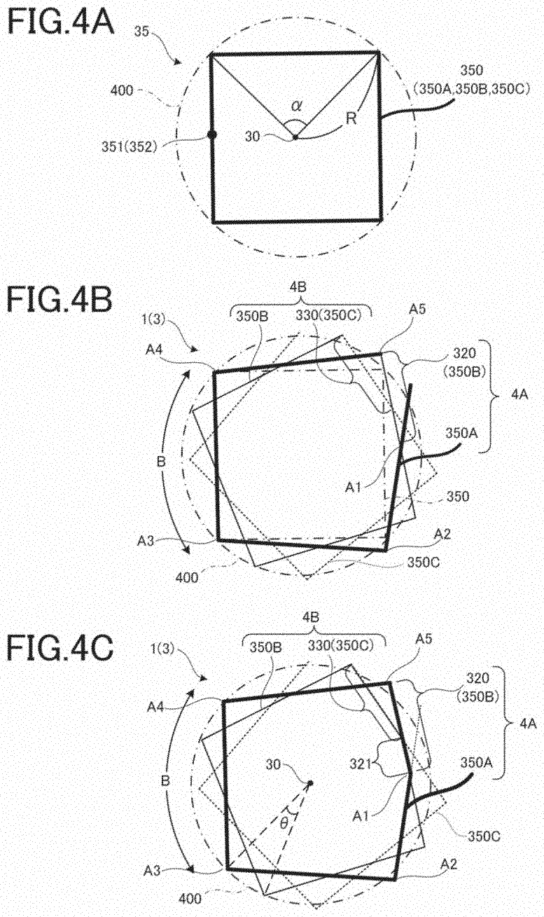

Referring to FIGS. 3A to 4C, a unit winding 4A will be described. FIG. 4A is a drawing, simplified for the sake of explanation, of the precursor 35 illustrated in FIG. 3C, when viewed from the axial direction A (axial direction E). FIGS. 4B and 4C are drawings, simplified for the sake of explanation, of a part of the helical body 3 illustrated in FIG. 2A, when viewed from the axial direction A (axial direction E).

As illustrated in FIGS. 3C and 4A, a precursor unit winding 350, which constitutes one winding of the precursor 35 in a state of winding the linear body 2 around the square columnar core material 5, is square shaped. As illustrated in FIG. 3B, a plurality of precursor unit windings 350 are contiguously aligned in the axial direction E of the precursor 35. The precursor unit windings 350, adjacent to each other in the axial direction E, of the precursor 35 contains an end point 351 of one of the adjacent precursor unit winding 350 and a start point 352 of another precursor unit winding 350 that approximately coincide. As a result, there is no phase difference in the shape of the precursor unit windings 350, adjacent to each other in the axial direction E. The axial direction E of the precursor 35 is the same as the axial direction A.

On the other hand, as illustrated in FIG. 4B, when the precursor 35 springs back and becomes the helical body 3, adjacent precursor unit windings 350A, 350B, and 350C expand. Thus, the precursor unit winding 350B, which is adjacent to the precursor unit winding 350A, rotates and shifts its posture (phase) with respect to the precursor unit winding 350A. Similarly, the precursor unit winding 350C, which is adjacent to the precursor unit winding 350B, rotates and shifts its posture (phase) with respect to the precursor unit winding 350B. When the helical body 3 is viewed from the axial direction A, the precursor unit windings 350A, 350B, and 350C are expanded, so each of the precursor unit windings 350A, 350B, and 350C does not have a closed shape by itself. At this time, when the helical body 3 is viewed from the axial direction A, the precursor unit winding 350A (the line connecting points A1, A2, A3, A4, and A5 in order) and a part 320 (the line connecting points A5 and A1) of the next contiguous precursor unit winding 350B form a closed winding shape. In other words, as illustrated in FIG. 4C, in the present embodiment, the winding shape is configured along the line connecting the points A1, A2, A3, A4, A5, and A1 in this order, and its outer shape becomes a pentagon with one vertex added to the precursor 35. The closed (annular) winding portion constitutes a unit winding 4A of the helical body 3, and its start and end points coincide with the point A1 at which the pair of adjacent precursor unit windings 350A and 350B intersect with each other.

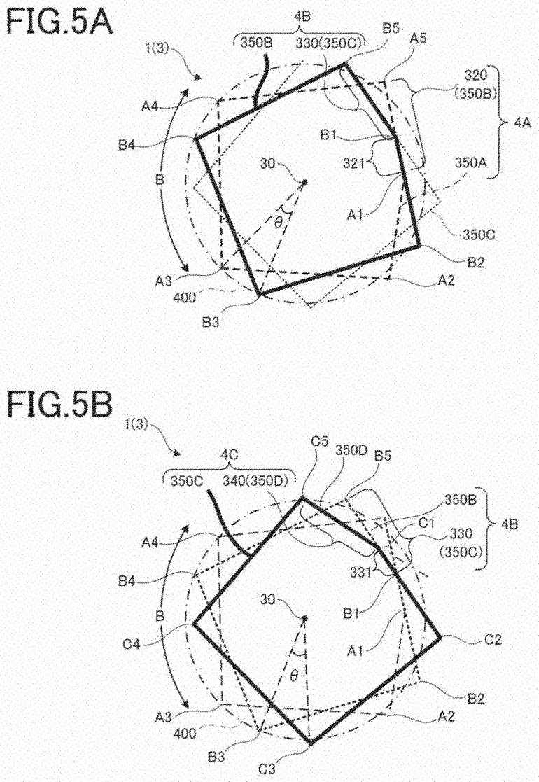

Similarly, as illustrated in FIG. 5A, the precursor unit winding 350B (the line connecting points B1, B2, B3, B4, and B5 in order) and a part 330 (the line connecting points B5 and B1) of the next contiguous precursor unit winding 350C form a closed winding shape. In other words, in the present embodiment, the winding shape is configured along the line connecting the points B1, B2, B3, B4, B5, and B1 in this order, and its outer shape becomes a pentagon with one vertex added to the precursor 35. The closed (annular) winding portion constitutes a unit winding 4B of the helical body 3, and its start and end points coincide with the point B1 at which the pair of adjacent precursor unit windings 350B and 350C intersect with each other.

In FIG. 5A, when the unit windings 4A and 4B are considered simultaneously, a part (terminal end part) on the side of a terminal end (rear end) of a final edge (the line connecting the points A5 and A1) of the former unit winding 4A and a part (start end part) on the side of a start end (front end) of a start edge (the line connecting points B1 and B2) of the latter unit winding 4B overlap with each other. Between winding shapes of the unit windings 4A and 4B, there is a phase difference θ (θ≠0 degree) to the extent that both unit windings 4A and 4B overlap at this overlapping part i.e. an overlap portion 321.

Furthermore, in the same manner, as illustrated in FIG. 5B, the precursor unit winding 350C (the line connecting points C1, C2, C3, C4, and C5 in order) and a part 340 (the line connecting the points C5 and C1) of the next contiguous precursor unit winding 350D form a closed winding shape. In other words, in the present embodiment, a unit winding 4C is configured along the line connecting the points C1, C2, C3, C4, C5, and C1 in this order. The pair of adjacent unit windings 4B and 4C form an overlap portion 331. The phase difference θ between the pair of unit windings 4B and 4C coincides with or approximates to the phase difference between the unit windings 4A and 4B.

As described above, the phase difference between the adjacent unit windings is assumed to be constant, but is not limited to this assumption and does not have to be constant. When the winding shape of the precursor unit windings 350A, 350B, and 350C is a regular polygon, the phase difference θ is preferably set so that the relationship with a central angle α of the regular polygon (0°<α<90°: refer to FIG. 4A) satisfies 0.1α≤θ≤0.9α, and more preferably satisfies 0.2α≤θ≤0.8α. Therefore, in the present embodiment, when a is 45 degrees, the phase difference θ is preferably set to satisfy 4.5 degrees≤θ≤40.5 degrees, and more preferably set to satisfy 9 degrees≤θ≤36 degrees.

In the present embodiment, the winding shape of the unit windings 4A and 4B is a pentagon, when the helical body 3 is viewed from the axial direction A. Accordingly, as illustrated in FIG. 2A, the winding shape of each of the unit windings 4A and 4B has vertex portions 41. Each vertex portion 41 is a region around a vertex 42 including the vertex 42 of the unit winding 4A or 4B and its vicinity. In FIGS. 4A and 5B, the vertices 42 correspond to the points A1, A2, A3, A4, A5, B1, B2, B3, B4, B5, C1, C2, C3, C4, and C5. When the winding shape of the unit windings 4A and 4B has a phase difference θ, as illustrated in FIGS. 2A and 4C, the vertex portions 41 shift in the circumferential direction B of the helical body 3 (hereinafter the circumferential direction of the helical body 3 will be referred to simply as circumferential direction.). As illustrated in FIG. 2B, the vertex portions 41 of the unit windings 4A to 4C are each arranged so as to sequentially shift to one side (counterclockwise) of the circumferential direction B along one side of the axial direction A. In other words, the unit windings 4A to 4C are in a state rotated by a predetermined angle relative to the respective adjacent unit windings 4A to 4C. The unit windings 4 are in a state sequentially rotated by the predetermined angle relative to the respective adjacent unit windings 4 in the same direction (one side of the circumferential direction B) along the one side of the axial direction A.

According to the configuration described above, as illustrated in FIG. 2A, when the unit windings 4A and 4B adjacent in the axial direction A are viewed from the axial direction A, the unit winding 4A has convex portions 40A (refer to the two-dotted chain area in FIG. 2A) that are convex outwardly in directions orthogonal to the axial direction A (hereinafter referred to as orthogonal directions) with reference to an outer edge 43B of the adjacent unit winding 4B. Similarly, when the unit windings 4B and 4C adjacent in the axial direction A are viewed from the axial direction A, the unit winding 4B has convex portions 40B (refer to the two-dotted chain area in FIG. 2A) that are convex outwardly in orthogonal directions with reference to an outer edge 43C of the adjacent unit winding 4C. Similarly, the unit winding 4C has convex portions 40C that are convex outwardly in orthogonal directions with reference to an outer edge 43D of the adjacent unit winding 4D. The convex portions 40A to 40C are configured to include the vertex portions 41 of the unit windings 4, as illustrated in FIG. 2A.

The linear body 2 is made of a material that allows the precursor 35 to spring back. Examples of such a material may include every type of metal having elastic and plastic regions. In particular, a platinum-tungsten alloy (Pt—W) is preferable as the metal, because of resistance to artifacts in X-ray CT and improvement in visibility in MRI.

In particular, when the unit winding 4 is in the shape of a regular polygon, and when the radius of a circumscribing circle 400 of the regular polygon that forms the precursor unit winding 350A (refer to FIG. 4A) is R and the number of corners of the regular polygon is n, the circumferential length A of an outer edge of the precursor unit winding 350A around a central axis 30 of the precursor unit winding 350A satisfies the following mathematical formula 1.

$$A = 2 R \times sin ( \frac{360 °}{2 n} ) \times n \qquad ( Formula 1 )$$

When the radius of the linear body 2 is w, the length B of a centerline of the linear body 2 as it completes one rotation cycle satisfies the following mathematical formula 2, where the linear body 2 constitutes the precursor unit winding 350A that goes around the central axis 30.

$$B = 2 \times \{ \frac{2 R \times sin ( \frac{360 °}{2 n} ) \times n}{2 π} - ω \} \times π \qquad ( Formula 2 )$$

The total amount εₚ of strain generated to the outer edge of the precursor unit winding 350A around the central axis 30 of the precursor unit winding 350A satisfies the following mathematical formula 3.

$${ɛ}_{p} = 100 \times [ \frac{A - B}{B} ] \qquad ( Formula 3 )$$

At this time, a corner strain amount εₘ generated to one corner of the precursor unit winding 350A satisfies the following mathematical formula 4.

$${ɛ}_{m} = 100 \times [ \frac{A - B}{B} ] \div n \qquad ( Formula 4 )$$

Substituting the mathematical formulas 1 and 2 into the mathematical formula 4, the following mathematical formula 5 is obtained.

$${ɛ}_{m} = 100 \times [ \frac{\begin{matrix}2 R \times sin ( \frac{360 °}{2 n} ) \times n - \\ 2 \times \{ \frac{2 R \times sin ( \frac{360 °}{2 n} ) \times n}{2 π} - ω \} \times π\end{matrix}}{2 \times \{ \frac{2 R \times sin ( \frac{360 °}{2 n} ) \times n}{2 π} - ω \} \times π} ] \div n \qquad ( Formula 5 )$$

The mathematical formula 5 is then rearranged into the following mathematical formula 6.

$${ɛ}_{m} = 100 \times \{ \frac{R \times sin ( \frac{360 °}{2 n} ) \times n}{R \times sin ( \frac{360 °}{2 n} ) \times n - πω} - 1 \} \div n \qquad ( Formula 6 )$$

If the maximum strain εₛ (%) of the linear body 2 is smaller than the corner strain amount εₘ (%), the linear body 2 may be torn off during manufacturing of the medical marker 1, or cracks may occur at corners of the medical marker 1. For this reason, the linear body 2 is preferably made of a material having a maximum strain εₛ (%) equal to or greater than the corner strain amount εₘ (%). In other words, the maximum strain εₛ (%) and the corner strain amount εₘ (%) satisfy the following mathematical formula 7.

$${ɛ}_{s} ≧ 100 \times \{ \frac{R \times sin ( \frac{360 °}{2 n} ) \times n}{R \times sin ( \frac{360 °}{2 n} ) \times n - πω} - 1 \} \div n \qquad ( Formula 7 )$$

The medical marker 1 configured as described above is formed with a plurality of convex portions arranged in the circumferential direction B and the axial direction A on its periphery. When the medical marker 1 is placed at a desired site in a living body, the convex portions are embedded in the site. As a result, the medical marker 1 does not easily come off from the site. In other words, the convex portions function as anchors to keep the medical marker 1 remaining at the desired site in the living body. In addition, if the phase difference θ in the winding shape is constant between the adjacent unit windings 4A and 4B, the convex portions are uniformly arranged in the circumferential direction B and the axial direction A, so no matter how the medical marker 1 is placed at the desired site in the living body, the convex portions are necessarily embedded in the site.

To reduce the amount of protrusion of the convex portion of the helical body 3 (which can be defined, for example, as the maximum protrusion in a radial direction of the latter unit winding with reference to an outer edge of the former unit winding), the phase difference θ between the adjacent unit windings 4 may be reduced. Conversely, to increase the amount of protrusion of the convex portion of the helical body 3, the phase difference θ between the adjacent unit windings 4 may be closer to 0.5α (α: the central angle of the polygon). In other words, the phase difference θ is determined from the viewpoint of sufficiently achieving the anchoring function of the convex portion.

In order to increase the number of the convex portions of the helical body 3, the number of vertices of a polygon may be increased. Conversely, to reduce the number of the convex portions of the helical body 3, the number of vertices of a polygon may be reduced. The number of the convex portions is also determined from the viewpoint of sufficiently achieving the anchoring function of the convex portions.

A maximum outer diameter D (refer to FIG. 2A) with reference to the central axis 30 of the helical body 3 as described above is preferably in the range of 0.1 to 1.5 (mm). This range is preferable because if the maximum outer diameter D is less than 0.1 (mm), the medical marker 1 is difficult to recognize by X-rays or MRI, and if the maximum outer diameter D is more than 1.5 (mm), a corresponding insertion needle becomes too thick and minimal invasiveness cannot be obtained.

The linear body 2 may be in the shape of an approximate circle or a polygon in cross section. When the linear body 2 is in the shape of a circle in cross section, a line diameter is preferably in the range of 0.01 to 0.5 (mm). This is because if the line diameter is less than 0.01 (mm), the medical marker 1 is difficult to recognize by X-rays or MRI, and if the line diameter is more than 0.5 (mm), the helical body 3 having the maximum outer diameter D described above is difficult to produce.

The length of the helical body 3 in the axial direction A is preferably in the range of 3 to 50 (mm). This is because if the length of the helical body 3 is less than 3 (mm), the medical marker 1 is difficult to recognize by X-rays or MRI, and if the length of the helical body 3 is more than 50 (mm), the medical marker 1 is too large to remain at a desired site in a living body.

The pitch of the helical body 3 is preferably three times or less the line diameter of the linear body 2, in order to make the medical marker 1 easily recognizable by X-rays or MRI.

### Modification of First Embodiment

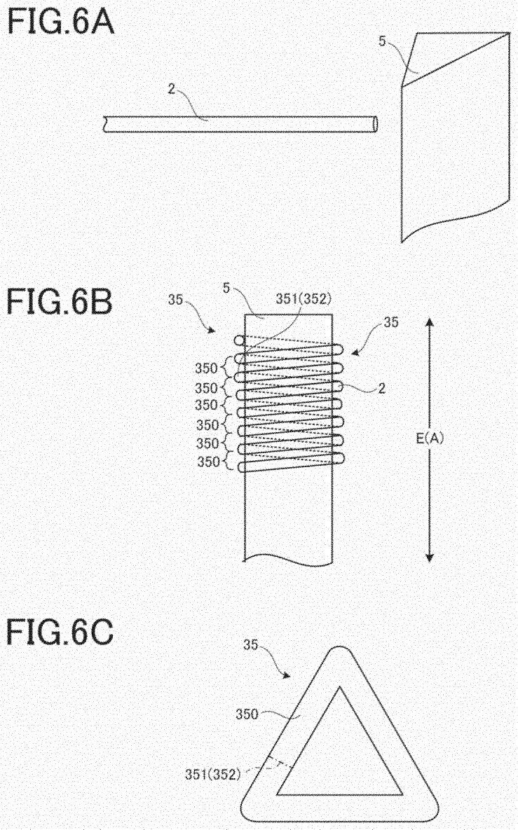

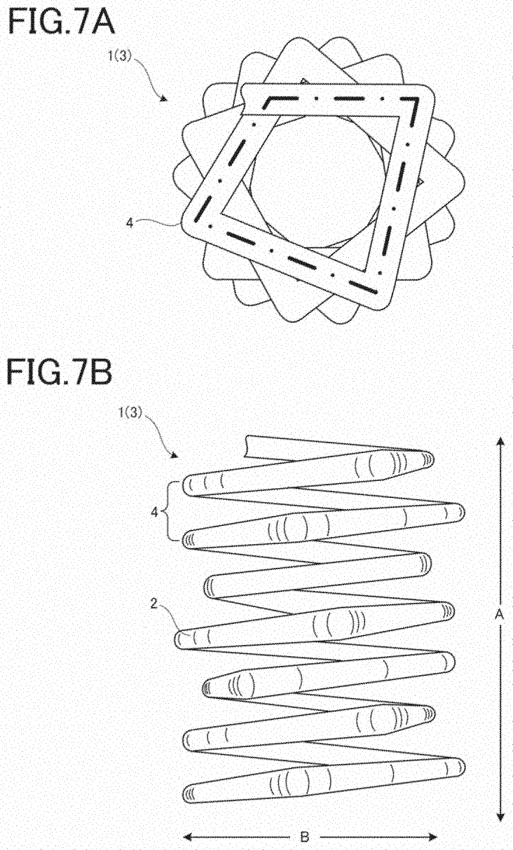

As a modification of the medical marker 1 according to the first embodiment of the present invention, mentioned are those in which the winding shape of unit windings 4 is a quadrangle, when a helical body 3 is viewed from its axial direction A. To produce the helical body 3 in which the winding shape of the unit windings 4 is a quadrangle, for example, as illustrated in FIG. 6A, a columnar core material 5 with the shape of a triangle in cross section and a linear body 2 are prepared. As illustrated in FIG. 6B, the linear body 2 is wound around the core material 5 into a helical shape. In this state, one winding of the linear body 2 (precursor unit winding 350) is shaped into a triangular shape to make a precursor 35 of the helical body 3 into a triangular prism shape. The precursor 35 is in the shape of a triangle, when the helical body 3 is viewed from the axial direction A, as illustrated in FIG. 6C. When the core material 5 is removed from the precursor 35, as illustrated in FIGS. 7A and 7B, the precursor 35 springs back and becomes the helical body 3 in which the winding shape of the unit windings 4 is a quadrangle (refer to the alternate long and short dash line in FIG. 7A).

Furthermore, to produce a helical body 3 in which the winding shape of unit windings 4 is a hexagon or more polygon, a linear body 2 may be helically wound around a core material 5 having the shape of a polygon in cross section, of which the number of corners is one less than the number of corners of the target polygon corresponding to the winding shape of the unit windings 4. When the core material 5 is removed from a precursor 35, the precursor 35 springs back and becomes the helical body 3 in which the winding shape of the unit windings 4 is a hexagon or more polygon.

In other words, in the medical marker 1 according to the present embodiment, the winding shape of the unit windings 4 should be a non-perfect circle, when the helical body 3 is viewed from the axial direction A. The non-perfect circle is a shape that excludes a perfect circle. The non-perfect circle has a vertex region (corresponding to a vertex portion 41 illustrated in FIG. 2A) at which the curvature is locally reduced. Therefore, the non-perfect circle includes not only a polygon but also a shape obtained by expanding to open a closed winding shape such as an ellipse, an egg shape, or a star shape, which will be described later, and a shape obtained by crushing the perfect circle.

One of the unit windings 4 adjacent in the axial direction A has a convex portion that is convex in the orthogonal direction from an outer edge of the other unit winding 4. The convex portion is composed of at least a part of a vertex region. As in a case in which the winding shape of the unit windings 4 is a pentagon, in a helical body 3, vertex regions (corresponding to vertex portions 41) of the unit windings 4 shift in the circumferential direction B of the helical body 3. The vertex regions (corresponding to the vertex portions 41) of the unit windings 4 are each arranged so as to sequentially shift to one side (counterclockwise) of the circumferential direction B along one side of an axial direction A.

By changing the cross-sectional shape of a core material 5, it is possible to manufacture helical bodies 3 having various winding shapes of unit windings 4, while the above-described conditions are satisfied. The winding shapes of the unit windings 4 may include, for example, a polygon or ellipse having a plurality of vertex regions, an egg shape having one vertex region (e.g., half is a regular arc and the remainder is an elliptical arc), and the like. The winding shape of the unit windings 4 of the helical body 3 as described above is also included in the range of the non-perfect circle.

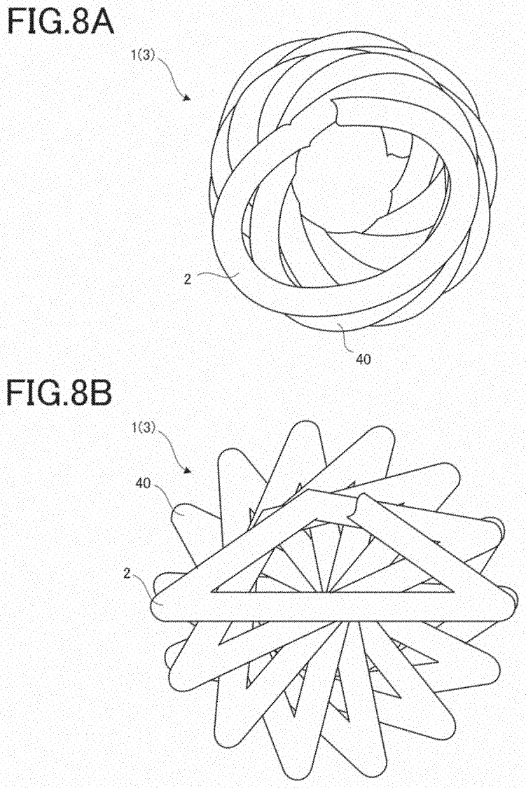

For example, in the case of using a columnar core material 5 with the shape of an ellipse in cross section, after making a precursor 35 by helically winding a linear body 2 around the core material 5, springing back the precursor 35 allows to form a helical body 3 that includes an expanded elliptical shape in part of the winding shape of the unit windings 4, as illustrated in FIG. 8A. Furthermore, in the case of using a columnar core material 5 having the shape of an isosceles triangle with a vertex angle of 90 degrees or more in cross section, after making a precursor 35 by helically winding a linear body 2 around the core material 5, springing back a precursor 35 allows to form convex portions 40 with a sharp vertex angle, as illustrated in FIG. 8B.

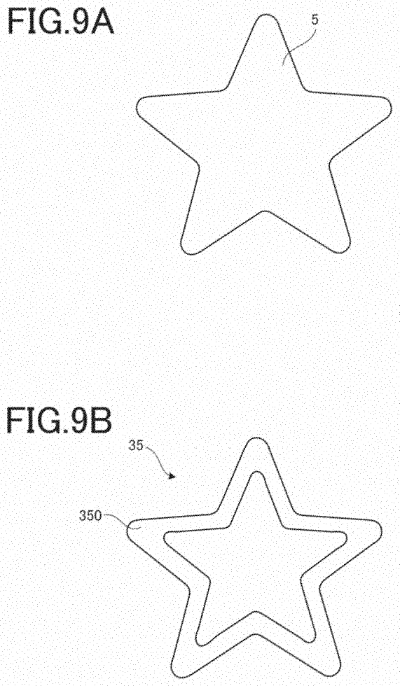

When the winding shape of unit windings 4 is a pentagon, the number of convex portions of a helical body 3 increases because the number of vertex portions 41 increases as compared to the case of a quadrangle. Furthermore, in the case of increasing the number of corners in the winding shape of unit windings 4, the number of convex portions of a helical body 3 increases. However, in order for the convex portions to function as anchors to remain the medical marker 1 at a desired site in a living body, the size of a vertex angle of the convex portion is preferably 135 degrees or less, and more preferably 108 degrees or less. This is because the smaller the size of the vertex angle of the convex portion, the more easily the convex portion is deeply embedded into the desired site in the living body. From this viewpoint, as illustrated in FIG. 9A, after making a precursor 35 by helically winding a linear body 2 around the core material 5 having the shape of a star in cross section, a helical body 3 is preferably made by springing back the precursor 35 (refer to FIG. 9B) that the winding shape is the shape of a star. The winding shape of the unit windings 4 of the helical body 3 as described above is also included in the range of the non-perfect circle. With such a helical body 3, the size of a vertex angle of convex portions can be reduced, even when the number of vertex regions is increased. From this viewpoint, a helical body manufactured using a core material 5 with the shape of a concave polygon such as a star in cross section can have higher anchoring function than a helical body manufactured using a core material 5 with the shape of a convex polygon in cross section. The cross-sectional shape of the core material 5 is preferably a non-perfect circle. In this case, the core material 5 can have any cross-sectional shape, as long as the cross-sectional shape is the non-perfect circle. When the coiling device is used to form the linear body 2 into a spiral body 3, the linear body 2 is preferably formed into the non-perfect circle coil. The non-perfect circle is a shape that excludes a perfect circle, as described above.

### Second Embodiment

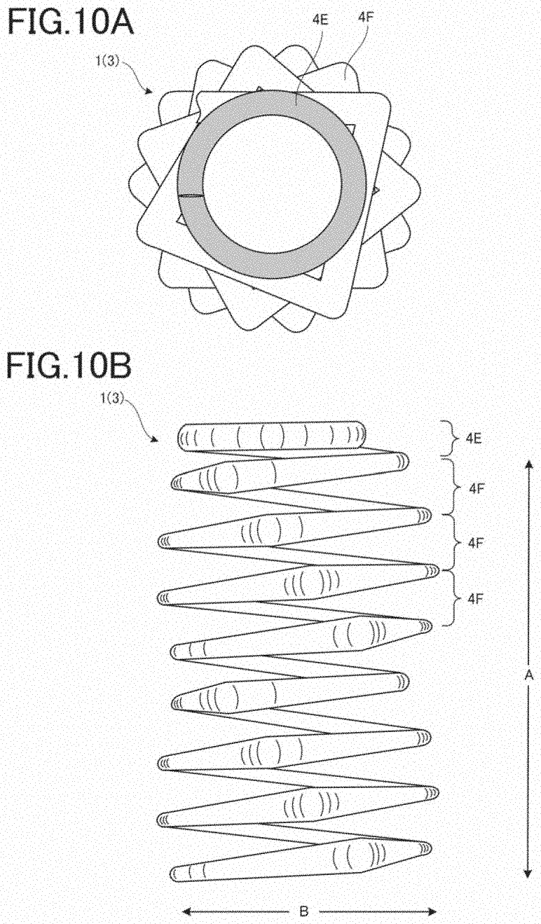

In a medical marker 1 according to a second embodiment of the present invention, as illustrated in FIGS. 10A and 10B, the winding shape (hereinafter referred to as end winding shape) of a unit winding 4E (hereinafter referred to as end unit winding) at an end of a helical body 3 in the axial direction A is different from the winding shape (hereinafter referred to as middle winding shape) of unit windings 4F (hereinafter referred to as middle unit windings) in the middle of the helical body 3 in the axial direction A. This is for the purpose of easily recognizing the positions of an end and middle of the medical marker 1 by X-ray or MRI. As a result, the posture of the medical marker 1 can be easily confirmed. In other words, the end unit winding 4E has a role in helping to recognize the position and posture of the helical body 3.

In FIGS. 10A and 10B, only the end winding shape of the end unit winding 4E at one end is different from the middle winding shape of the middle unit winding 4F. However, the end winding shape of an end unit winding at the other end (not illustrated) may also be different from the middle winding shape of the middle unit winding 4F. Also, the end winding shape of the end unit winding at the other end (not illustrated) may be different from the end winding shape of the end unit winding 4E at the one end. When the end winding shapes of the end unit windings at both ends of the helical body 3 are different from each other, the position of the ends of the medical marker 1 and the posture of the medical marker 1 can be confirmed more reliably by X-rays or MRI.

In FIG. 10A, the end winding shape of the end unit winding 4E is a perfect circle, and the middle winding shape of the middle unit windings 4F is a quadrangle (refer to FIG. 7A). However, the end winding shape of the end unit winding 4E may be any shape other than a perfect circle, as long as the end winding shape is different from the middle winding shape of the middle unit winding 4F.

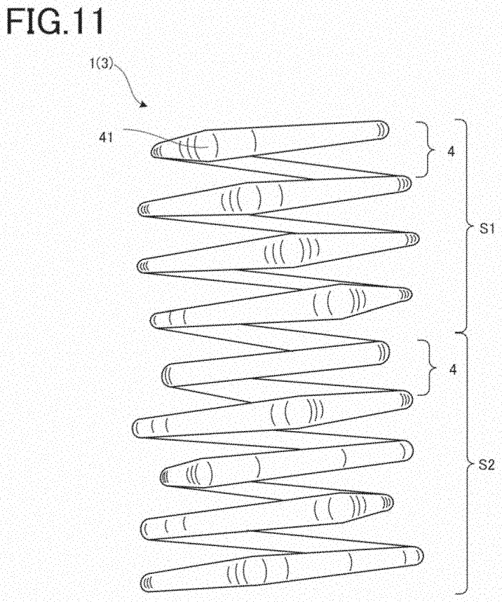

In order to confirm the position of an end of the medical marker 1 and the posture of the medical marker 1, the winding shape of the unit windings 4 may be different for each section in the axial direction A in addition to the above features. There may be at least two sections in the axial direction A. For example, as illustrated in FIG. 11, in a section S1 in the axial direction A, the winding shape of the unit windings 4 is a pentagon (refer to FIG. 2A), and in a section S2 that is contiguous to the section S1 in the axial direction A, the winding shape of the unit windings 4 is a quadrangle (refer to FIG. 7A). The winding shapes of the unit windings 4 should be different at least in the adjacent sections, and the winding shapes of the unit windings 4 may be the same or different in non-adjacent sections.

The aspects of the medical marker 1 described above may be applied to a coil portion of a catheter of which the tip is constituted of the coil portion. In this case, the coil portion of the catheter is configured in the same manner as the medical marker 1 according to the above-described embodiments. The surface of the coil portion of the catheter is preferably coated with a predetermined resin in order to improve adhesion with other resin medical members.

The medical marker 1 according to the present invention is not limited to the above-described embodiments, and as a matter of course, various modifications can be added within the scope not deviating from the gist of the present invention.

What is claimed is:

1. A medical marker comprising:
a helical body configured by a linear body in a helical shape, wherein
when the helical body is viewed from an axial direction of the helical body, a winding shape of a unit winding constituting one winding of the helical body is a non-perfect circle, and
a plurality of the unit windings contiguously aligned in the axial direction overlap at a terminal end part of one of the adjacent unit windings and a start end part of the other of the adjacent unit windings, so that there is a phase difference in the winding shape by an amount of overlap.

2. The medical marker according to claim 1, wherein the winding shape of the unit winding has a vertex region with a locally reduced curvature.

3. The medical marker according to claim 2, wherein the winding shape of the unit winding is any one of a polygon with a plurality of the vertex regions and a crushed perfect circle.

4. The medical marker according to claim 2, wherein the vertex regions of the plurality of unit windings contiguously aligned in the axial direction are each arranged so as to sequentially shift to one side of a circumferential direction of the helical body along one side of the axial direction.

5. The medical marker according to claim 1, wherein when the helical body is viewed from the axial direction, an end winding shape of an end unit winding constituting one winding of the helical body at least at one end in the axial direction is different from the winding shape of the unit winding in the middle of the helical body in the axial direction.

6. The medical marker according to claim 5, wherein the end winding shape is a perfect circle.

7. The medical marker according to claim 1, wherein when the unit winding is in the shape of a regular polygon, the phase difference θ and a central angle α of the regular polygon satisfy 0.1α≤θ≤0.9α.

8. The medical marker according to claim 1, wherein when the unit winding is in the shape of a regular polygon, a maximum strain εₛ of a material constituting the helical body, a radius R of a circumscribing circle of the regular polygon, a radius ω of the linear body, and the number n of corners of the regular polygon satisfy the following mathematical formula:
$${ɛ}_{s} ≧ 100 \times \{ \frac{R \times sin ( \frac{360 °}{2 n} ) \times n}{R \times sin ( \frac{360 °}{2 n} ) \times n - πω} - 1 \} \div n .$$

9. A method for manufacturing a medical marker having a helical body obtained by making a linear body in a helical shape, the method comprising:
forming the helical body by springing back, after the linear body is wound around a columnar core material,
when the helical body is viewed from an axial direction of the helical body, a winding shape of a unit winding constituting one winding of the helical body is a non-perfect circle, and
a plurality of the unit windings contiguously aligned in the axial direction overlap at a terminal end part of one of the adjacent unit windings and a start end part of the other of the adjacent unit windings, so that there is a phase difference in the winding shape by an amount of overlap.

10. A method for manufacturing a medical marker having a helical body obtained by making a linear body in a helical shape, the method comprising:
forming the helical body by springing back, after the linear body is formed into a coil using a coiling device for forming the linear body into the coil,
when the helical body is viewed from an axial direction of the helical body, a winding shape of a unit winding constituting one winding of the helical body is a non-perfect circle, and
a plurality of the unit windings contiguously aligned in the axial direction overlap at a terminal end part of one of the adjacent unit windings and a start end part of the other of the adjacent unit windings, so that there is a phase difference in the winding shape by an amount of overlap.

11. The method for manufacturing the medical marker according to claim 9, wherein the helical body is shaped by heat treatment after being spring-backed.

12. The method for manufacturing the medical marker according to claim 10, wherein the helical body is shaped by heat treatment after being spring-backed.

13. A medical marker comprising:
a helical body obtained by making a linear body in a helical shape, wherein
when the helical body is viewed from an axial direction of the helical body, at least one of a plurality of unit windings, each of which constitutes one winding of the helical body and which are contiguously aligned in the axial direction, has a convex portion that is convex outwardly in a direction orthogonal to the axial direction with reference to an outer edge of adjacent one of the unit windings.
